(19) United States
(12) Patent Application Publication
(10) Pub. No.: US 2008/0033570 A1
(43) Pub. Date: Feb. 7, 2008

(54) PROSTATIC STENT PLACEMENT DEVICE

(76) Inventors: Benjamin T. Blitz, Bloomington, IN (US); John H. Ward, Spencer, IN (US)

Correspondence Address: BRINKS HOFER GILSON & LIONE/CHICAGO/COOK, PO BOX 10395, CHICAGO, IL, 60610, US

(21) Appl. No.: 11/733,420

(22) Filed: Apr. 10, 2007

Related U.S. Application Data
(63) Continuation-in-part of application No. 10/899,807, filed on Jul. 27, 2004.
(60) Provisional application No. 60/791,591, filed on Apr. 12, 2006.
(60) Provisional application No. 60/491,781, filed on Aug. 1, 2003.

(51) Int. Cl. A61F 2/82; A61F 2/84

(52) U.S. Cl. 623/23.7; 604/103.05

(57) ABSTRACT

The present invention provides a method and apparatus for maintaining urine flow during and after urological procedures. The invention includes a stent for long term insertion into a patient's urinary tract, a Foley catheter which is used to support the stent during placement, a positioning shaft which is used to position the stent with respect to the catheter, and a protective sheath that retains these components in place during insertion of the stent. Alternatively, a Foley catheter with two balloons may be used in place of the stent positioner, with the stent being accurately positioned with one of the two balloons being surrounded by the stent.

### CROSS REFERENCE TO RELATED APPLICATIONS

This application claims priority to U.S. Provisional Application No. 60/791,591, filed on Apr. 12, 2006, and is hereby fully incorporated by reference herein. This application is additionally a continuation-in-part of U.S. application Ser. No. 10/899,807, filed on Jul. 27, 2004, which claims benefit of Provisional Application No. 60/491,781, filed on Aug. 1, 2003, both of which are hereby fully incorporated by reference herein.

### FIELD OF THE INVENTION

The present invention relates to the field of catheters and stents for use in a medical situation.

### BACKGROUND OF THE INVENTION

Several methods and apparatuses for maintaining urethral patency are known for use during and after urological treatments. For example, it is known in the art to use a Foley catheter that is positioned within the urethra with a balloon on the distal end that extends into the bladder with the catheter including a lumen to allow for urine flow through the lower urinary tract and to open an obstructed urethra.

It is also known in the art to provide an indwelling urethral catheter with a Foley-type balloon at the distal end of the catheter and a substantially non-compliant balloon lead shaft proximate to the Foley-type balloon to allow for urine flow through the lower urinary tract. This type of apparatus is discussed in U.S. Pat. No. 4,432,757. Similarly, U.S. Pat. No. 5,785,694 discloses an internal urinary catheter with a distal balloon that is positioned within the bladder when the catheter is inserted and prevents withdrawal of the catheter, and a second urethral retention balloon spaced toward the outlet end from the distal balloon to prevent the catheter from migrating further into the bladder.

It is also known in the art to use a urethral stent to maintain urethral patency after mildly invasive treatments for benign prostatic hyperplasia (“BPH”) or to maintain a flow path through the urethra after other lower urinary tract symptoms (LUTS). For example, The Spanner™ Temporary Prostatic Stent is a known apparatus that may be temporarily positioned within the urethra to enhance urine flow through the lower urinary tract. The Spanner™ is discussed in a publication titled “New Prostatic Stent for the Relief of Severe Lower Urinary Tract Symptoms,” by Alberto P. Corica, found at www.bbriefings.com/pdf/33/gs031_t_abbeym.pdf on Aug. 25, 2005. This stent is physically connected to a distal balloon, or other type of anchor on the distal end of the apparatus with connecting sutures, which prevents the stent from being permanently retained within the urethra. The Spanner™ is only designed to be temporarily inserted into the urethra.

While the use of a Foley catheter and The Spanner™ have been successfully used to promote urine flow through the urinary tract after urological procedures, these devices cannot be continuously inserted into an active patient, but usually require medical attention to the patient when inserted. Therefore, it is desired to have a device that can be implanted into a patient that provides for urethral patency after urological treatments that can be permanently inserted into the patient and require a minimal amount of monitoring. Additionally, it is desired to provide a simple and effective method of precisely implanting the permanent device into the patient.

### BRIEF SUMMARY

The present invention provides a method for inserting a stent into a patient. The method includes the steps of inserting a stent into a sheath, inserting a catheter and cylindrical member into the sheath until the catheter extends through a lumen in the stent and the distal end of the cylindrical member contacts a proximal end of the stent. Next, the cylindrical member is positioned within the sheath so a distal end of the stent is rearward of a balloon on the catheter. Next, the sheath, the catheter, the stent, and the cylindrical member are inserted into a patient. At this point, the balloon is inflated, the stent is positioned within the patient, and the sheath and cylindrical member are selectively withdrawn from the patient.

The present invention also provides a stent positioning device. The stent insertion device includes a catheter formed with a distal end and a proximal end, a balloon positioned on the distal end, and a hollow stent with a distal end and a proximal end, with the stent surrounding the catheter and positioned rearwardly of the balloon. The device also includes a stent positioner substantially surrounding the catheter and positioned rearward of a proximal end of the stent and a sheath surrounding the stent, the stent positioner, and the majority of the catheter.

The present invention additionally provides another stent positioning device. The stent insertion device includes a catheter including a distal end and a proximal end, a first balloon positioned on the distal end and a second balloon positioned proximally of the first balloon along a longitudinal axis of the catheter. A stent is provided with a distal end and a proximal end. The stent surrounds the second balloon of the catheter when the stent positioning device is assembled. A sheath surrounds the stent and the majority of the catheter when the stent positioning device is assembled.

Advantages of the present invention will become more apparent to those skilled in the art from the following description of the preferred embodiments of the invention that have been shown and described by way of illustration. As will be realized, the invention is capable of other and different embodiments, and its details are capable of modification in various respects. Accordingly, the drawings and description are to be regarded as illustrative in nature and not as restrictive.

### BRIEF DESCRIPTION OF THE DRAWINGS

FIG. 1 is a perspective view of one embodiment of a stent placement device.

FIG. 1a is an exploded view of the components of the stent placement device of FIG. 1.

FIG. 2 is a perspective view of the device of FIG. 1, showing all the components the stent placed within the sheath.

FIG. 3 is a perspective view of the stent placement device of FIG. 1, showing the sheath removed.

FIG. 4 is a perspective view of the stent positioner of the device of FIG. 1.

FIG. 5 is a perspective view of the stent of the device of FIG. 1.

FIG. 6 is a perspective view of the stent pusher of the device of FIG. 1.

FIG. 7 is a perspective view of the sheath of the device of FIG. 1.

FIG. 7a is a perspective view of the sheath of the device of FIG. 1, showing the arms cuffed.

FIG. 8 is a perspective view of the catheter of the device of FIG. 1.

FIG. 8a is a perspective view of the catheter, stent positioner, and a clip of the device of FIG. 1.

FIG. 9 is a perspective view of the penile meatus dilator and penile meatus sheath.

FIG. 10 is a flowchart of the method used with the device of FIG. 1.

FIG. 11 is a perspective view of a catheter used in a second embodiment of the stent placement device.

FIG. 12 is a flowchart of the method used with the device of FIG. 11.

### DETAILED DESCRIPTION

The foregoing detailed description is fully described for use in a male patient's urethra and bladder, with insertion through the patient's penile meatus. As can be understood by those of ordinary skill in the art after fully reviewing this disclosure, the embodiments discussed below can be successfully be used precisely position a stent in other lumens within a patient that can be directly accessed through an external aperture. Accordingly, while this application only discuses the use within a male patient's urethra for the sake of brevity, it should be understood that the invention can be used in other appropriate medical procedures.

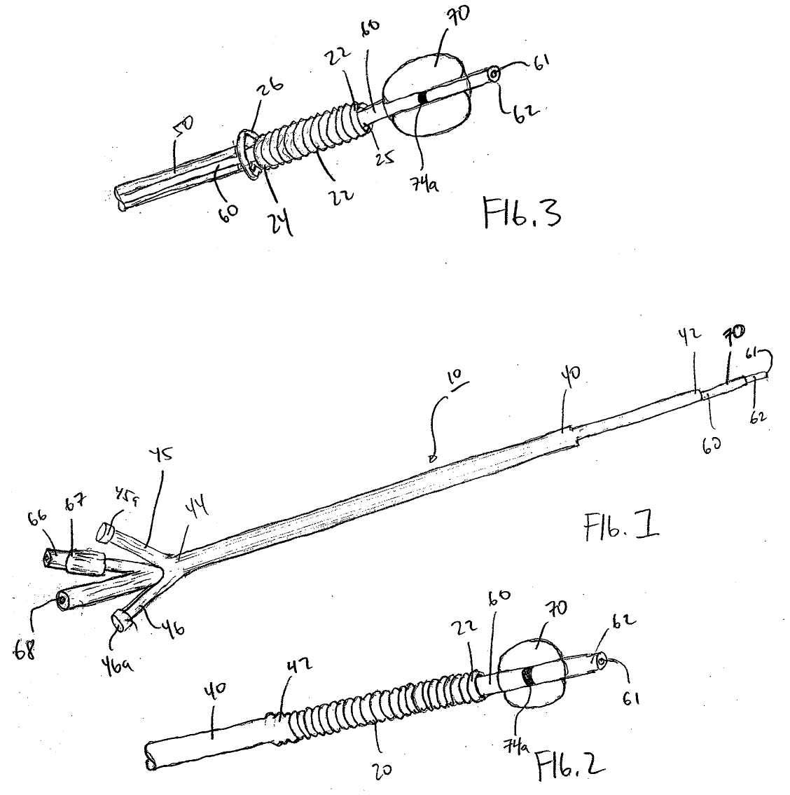

With reference to FIGS. 1-10, a stent placement device 10 for precise placement of a stent 20 within a patient is provided. The stent placement device 10 is useful in treatment during and after many urological procedures. Specifically, the stent placement device 10 is useful to properly position a stent 20 within a patient after mildly invasive treatments for benign prostatic hyperplasia (“BPH”).

The stent placement device 10 includes a stent 20, a stent pusher 30, a outer sheath 40, a stent positioner 50, and a balloon catheter 60. When the components are assembled for placement of stent 20 within the patient, the stent 20, stent positioner 50, and balloon catheter 60 are each inserted within an internal volume of outer sheath 40. Additionally, balloon catheter 60 is inserted into a partial cavity 56 of the stent positioner 50 and balloon catheter 60 is inserted through lumen 25 of stent 20. As discussed below with the method for accurately placing stent 20 into a selected position, stent 20 is the only member of stent placement device 10 that remains within the patient after the procedure for BPH is completed and the patient's urine is free of blood.

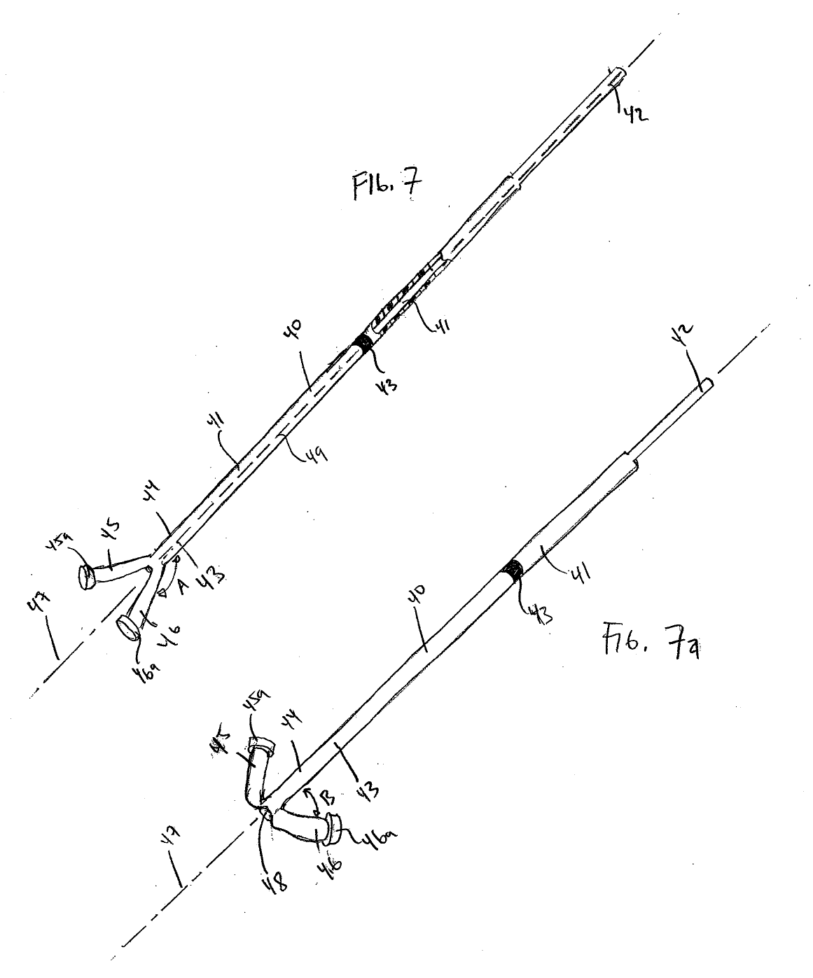

As best seen in FIGS. 3 and 5, stent 20 includes a distal end 22, a proximal end 24, and a lumen 25. Stent 20 is formed with a wound serpentine pattern that is preferably wound to achieve a constant outer diameter and a constant inner diameter with a lumen 25. Lumen 25 allows a fluid to flow through stent 20 when inserted into a patient. Stent 20 may be selectively used to maintain flow in many medical procedures. For example, stent 20 may be inserted into a urethra to maintain patency and to provide a urine flow path from the bladder through the urethra to the outside, specifically during and after mildly invasive procedures for BPH.

Proximal end 24 of stent 20 is formed with a flare 26. In some embodiments, the flare 26 may have a diameter of approximately 55 Fr while the outer diameter of the remainder of stent 20 is approximately 21 Fr. In other embodiments, stent 20 can be manufactured with other sizes and diameters depending on the desired use of stent 20. Proximal end 24 of stent 20 includes a flare 26 that is a greater diameter than the outer diameter of the remaining portions of stent 20. This provides stent 20 with an extended surface that maintains contact with the walls of the urethra, or other flow path into which stent 20 is inserted, to maintain the stent 20 in position to provide for unobstructed flow. Stent 20 is manufactured from a material that allows stent 20 to be flexible enough to be inserted through a curved flow path, such as a patient's urethra, while being formed with sufficient radial strength to maintain the flow path through the urethra unobstructed to allow sufficient urine flow.

As best shown in FIG. 6, stent pusher 30 is formed as a long flexible cylinder and is insertable into a lumen 41 formed within outer sheath 40 through the aperture 48 on proximal end 44 of outer sheath 40. Stent pusher 30 has a wider outer diameter than the inner diameter of stent 20 to allow distal end 34 of stent pusher 30 to contact flare 26 on proximal end 24 (with flare 26 reduced, as discussed below) so that forward movement of stent pusher 30 corresponding forward movement of the stent 20. As discussed below, stent pusher 30 selectively moves stent 20 longitudinally within outer sheath 40 toward distal end 42 of outer sheath 40. After stent pusher 30 has positioned stent 20 within outer sheath 40, stent pusher 30 is withdrawn through proximal end 44 of the outer sheath 40 and discarded. In some embodiments, stent pusher 30 may have a tapered distal end 32, formed similarly to an amplatz dilator.

As discussed below, stent pusher 30 may have a positive stop 36 formed with a larger circumference than the inner diameter of outer sheath 40 (discussed below) to selectively positioned stent 20 within sheath 20. In other embodiments, stent pusher 30 may have an index mark provided thereon to aid the medical professional in precisely placing stent 20 within the outer sheath 40.

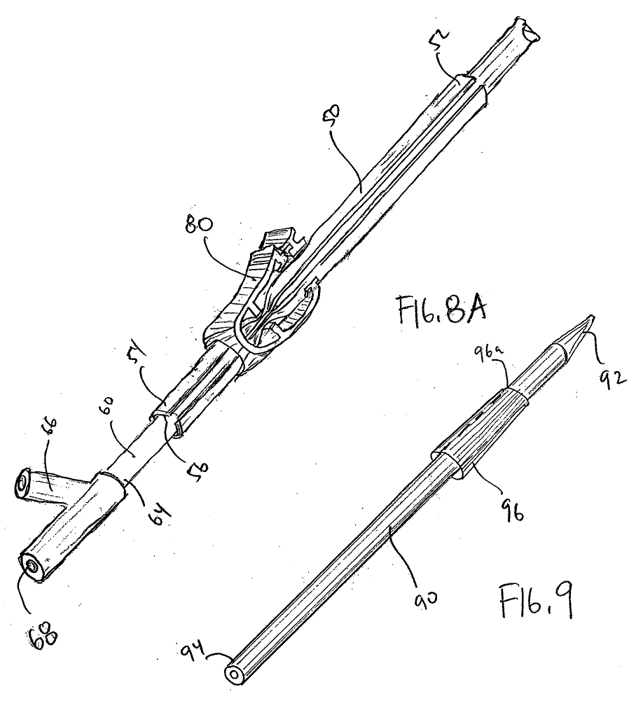

As best shown in FIG. 7, outer sheath 40 is provided for use with stent positioning device 10. Outer sheath 40 is formed as a hollow sleeve, forming lumen 41, to retain stent 20 in position with respect to the remaining members of stent positioning device 10. This allows for accurate placement of stent 20 within the desired flow channel. Outer sheath 40 includes distal end 42 and proximal end 44. Proximal end 44 includes an aperture 48 that allows stent 20, stent pusher 30, stent positioner 50, and balloon catheter 60 to be slidingly inserted through lumen 41 (FIG. 7) of outer sheath 40.

Preferably, outer sheath 40 is formed with a weakened region 49 that extends along the longitudinal axis 47 of outer sheath 40. Outer sheath 40 is formed with two arms 45, 46 that extend from proximal end 44 and are separated from each other along weakened region 49. Arms 45, 46 are provided to give the medical practitioner a surface to manipulate to tear outer sheath 40 along weakened region 49, to allow for removal of outer sheath 40 from the patient, as discussed below. In some embodiments, arms 45, 46 are formed with thumbscrews, or similar structures, that provide an ergonomic surface for the medical professional to manipulate to aid in splitting outer sheath 40 when arms 45, 46 are pulled apart. As can be understood, splitting outer sheath 40 along weakened region 49 allows outer sheath 40 to be removed from the patient without removing the other components of the stent positioning device 10 from the patient.

In the embodiment shown in FIG. 7, the weakened region 49 includes one or a plurality of perforations along the longitudinal axis 47 of outer sheath 40. In other embodiments, the sheath can be scored with a single or continuous score or have a thinner portion along the weakened region 49 to aid in the removal of outer sheath 40 from stent placement device 10 when it is inserted into a patient. Preferably, outer sheath 40 includes two weakened regions 49 on opposite sides of to allow outer sheath 40 to be torn into two sections. In other embodiments, outer sheath 40 can include only one weakened region 49.

In some embodiments, a radiopaque marker 43 may be provided on outer sheath 40. Radiopaque marker 43 provides the physician with an indication of the location of outer sheath 40 (and therefore stent 20 and the remaining components of stent positioning device 10) within the patient after stent positioning device 10 is inserted. Radiopaque marker 43 may be formed from radioactive platinum iridium, echo-tipped radiopaque stainless steel, UTT, or other types of radiopaque markers that are known to those of ordinary skill in the art.

After stent positioning device 10 is inserted into the patient, the physician can monitor the position of the device 10, and specifically the stent 20, within the patient using various methods known in the art. For example, a transrectal ultrasound probe is often inserted into the patient's anus during urological procedures used with radiopaque markers. The probe detects the radiation produced by radiopaque marker 43 and gives the physician an independent indication of the position of the marker 43 and the components of stent positioning device 10.

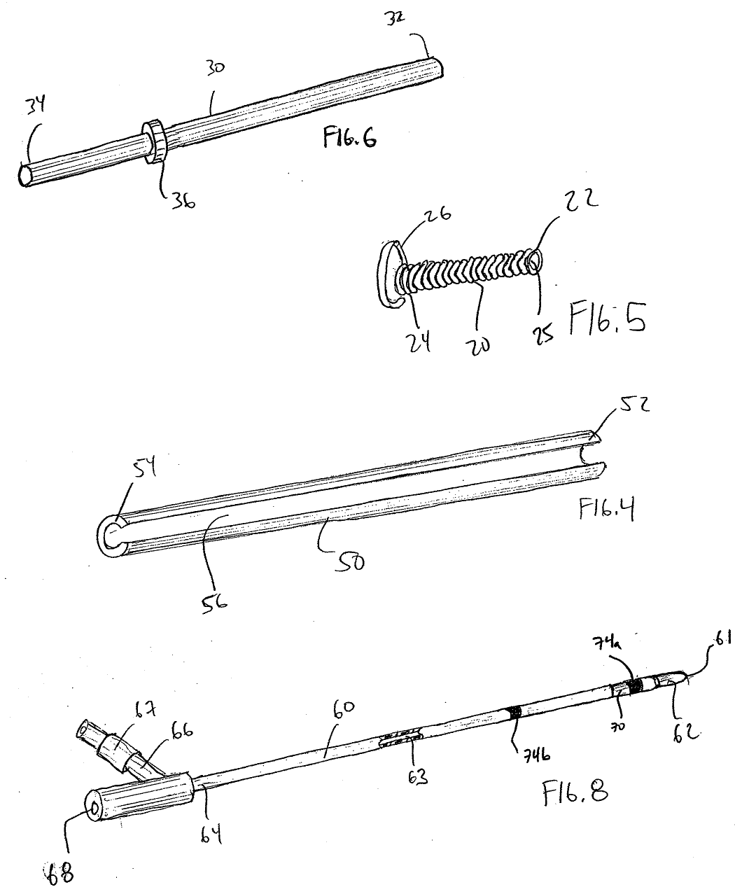

As best shown in FIG. 4, stent positioner 50 is provided for use with stent positioning device 10. Stent positioner 50 is formed as a long thin member, and may be formed with a C-shaped cross-section. Stent positioner 50 includes distal end 52 and proximal end 54. Stent positioner 50 has an outer diameter that is less than the inner diameter of sleeve 40 to allow stent positioner 50 to be inserted within the lumen 41 of sleeve 40. Additionally, stent positioner 50 includes an inner diameter, within a cavity 56, that is greater than the outer diameter of balloon catheter 60 to allow balloon catheter 60 to be inserted within the cavity 56, as best shown in FIG. 3.

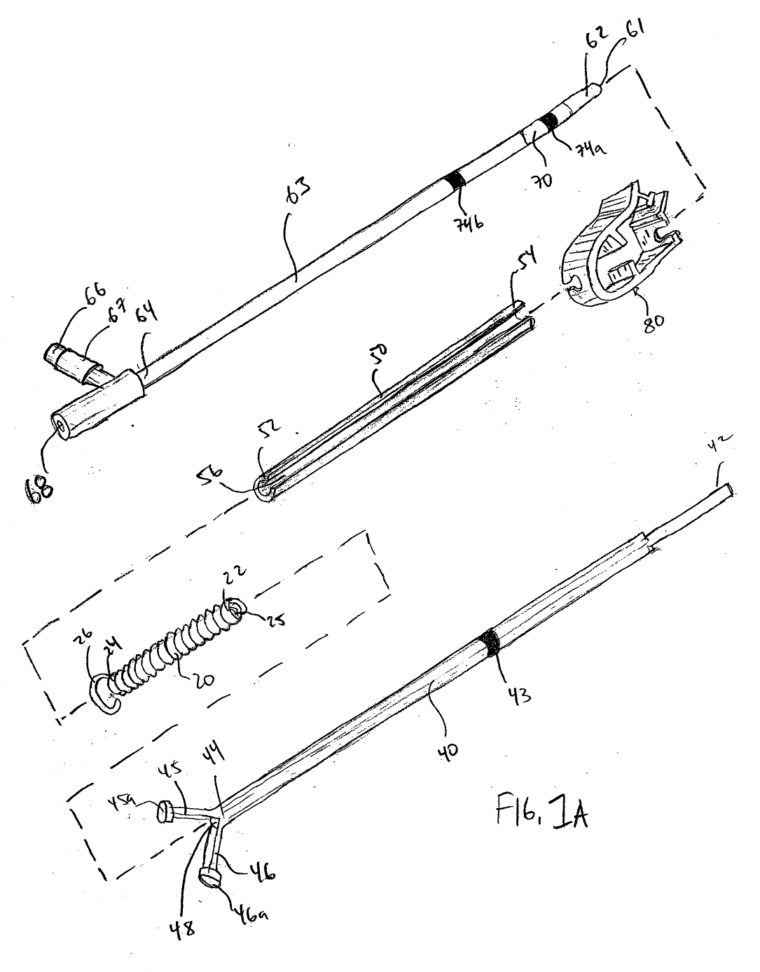

As shown in FIG. 8, a balloon catheter 60 is provided for assembly in stent positioning device 10. Preferably, balloon catheter 60 is a Foley-type balloon catheter. Balloon catheter 60 is formed as a long thin member with distal end 62 and proximal end 64. Balloon catheter 60 is formed with a lumen 63 (FIG. 8) that extends along the length of balloon catheter 60 from a distal port 61 to a drainage port 68. Distal end 62 includes a balloon 70 that can be selectively inflated with a working fluid (as shown in FIGS. 2 and 3) and selectively deflated (as shown in FIG. 1).

Proximal end 64 is formed with inflation/deflation port 66 and drainage port 68. Inflation/deflation port 66 may extend from the balloon catheter 60 at an oblique angle from the longitudinal axis of balloon catheter 60, in some embodiments forming an obtuse angle with respect to the length of balloon catheter 60 from proximal end 64 to distal end 62. Inflation/deflation port 66 may additionally include an internal check valve 67 located within inflation/deflation port 66 to prevent flow of the working fluid out of balloon catheter 60 through inflation port 66. Inflation/deflation port 66 is connected to balloon 70 through a lumen (not shown) to provide a flow path for the working fluid from the inflation/deflation port 66 to balloon 70. The lumen is preferably formed inside flexible tubing. Inflation/deflation port 66 is preferably a slip fit valve or a Luer lock style valve to accept a source of working fluid from a syringe or other pressurized fluid source as is known in the art. Alternatively, port 66 may be formed as a solid plug that is opened with a syringe to inflate or deflate balloon 70.

Balloon catheter 60 additionally includes drainage port 68, which is positioned at proximal end 64 of balloon catheter 60. Balloon catheter 60 preferably includes lumen 63 extending through the length of balloon catheter 60 to the inlet 61. Balloon catheter 60 and balloon 70 are preferably formed from silicone. In other embodiments, balloon catheter 60 may be formed from other materials that provide the strength and hardness to include internal lumen 63 while allowing balloon catheter 60 to be sufficiently bent while it is inserted through the patient's urethra (or other selected portion of the patient) and positioned within the patient's bladder.

In some embodiments, one or more radiopaque markers may be provided on balloon catheter 60. As discussed above with respect to outer sheath 40, radiopaque markers provided on balloon catheter 60 allow the physician to determine that balloon catheter 60 is properly inserted into the patient, and that stent 20 is properly positioned. Radiopaque markers made from radioactive platinum iridium, Echo-tipped radiopaque stainless steel, UTT, and other types of radiopaque markers known to those of ordinary skill in the art may be used.

As shown in FIG. 8, a first radiopaque marker 74a may be proved under balloon 70 and/or a second radiopaque marker 74b may be provided rearward of first radiopaque marker 74a on balloon catheter 60. In some embodiments, second radiopaque marker 74b is provided 9 cm rearward of the proximal side of balloon 70, which corresponds to the length of stent 20. In other embodiments, second radiopaque marker 74b may be provided at other distances from balloon 70. The position of the radiopaque markers may be detected by a transrectal ultrasound probe as discussed above, or by other methods known to those of ordinary skill in the art.

As shown in FIG. 9, a penile meatus dilator 90 and sheath 96 are provided. Penile meatus dilator 90 is a cylindrical shaft with a distal end 92 that is formed as a tapered end and a proximal end 94. Penile meatus sheath 96 is hollow and forms a lumen 98 through the length of sheath 96. Distal aperture 96a of penile meatus sheath 96 has substantially the same inner diameter as the outer diameter of penile meatus dilator 90 to allow penile meatus sheath 96 to fit over penile meatus dilator 90, but only slide with respect to dilator 90 when pushed with respect to penile meatus dilator 90.

Penile meatus dilator 90 may be used by the physician prior to inserting stent positioning device 10 into the penile meatus of the patient. On many occasions, it is difficult to insert medical devices through the patient's penile meatus and urethra. Penile meatus dilator 90 includes tapered distal end 92 that may be easily inserted into the patient's penile meatus to gradually expand the urethra within the penile meatus. After distal end 92 is inserted into the penile meatus, penile meatus sheath 96 is positioned within the penile meatus and penile meatus dilator is removed. Penile meatus sheath maintains the urethral aperture open to allow stent positioning device 10 to be inserted into the patient. After stent 20, stent positioner 50, and balloon catheter 60 are inserted and positioned within the patient (as discussed in detail below), the penile meatus sheath 96 may be removed from the patient and discarded.

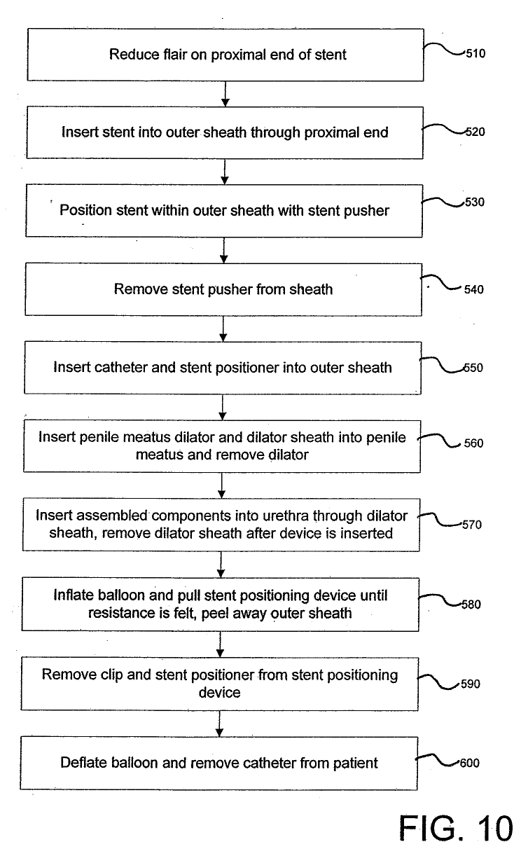

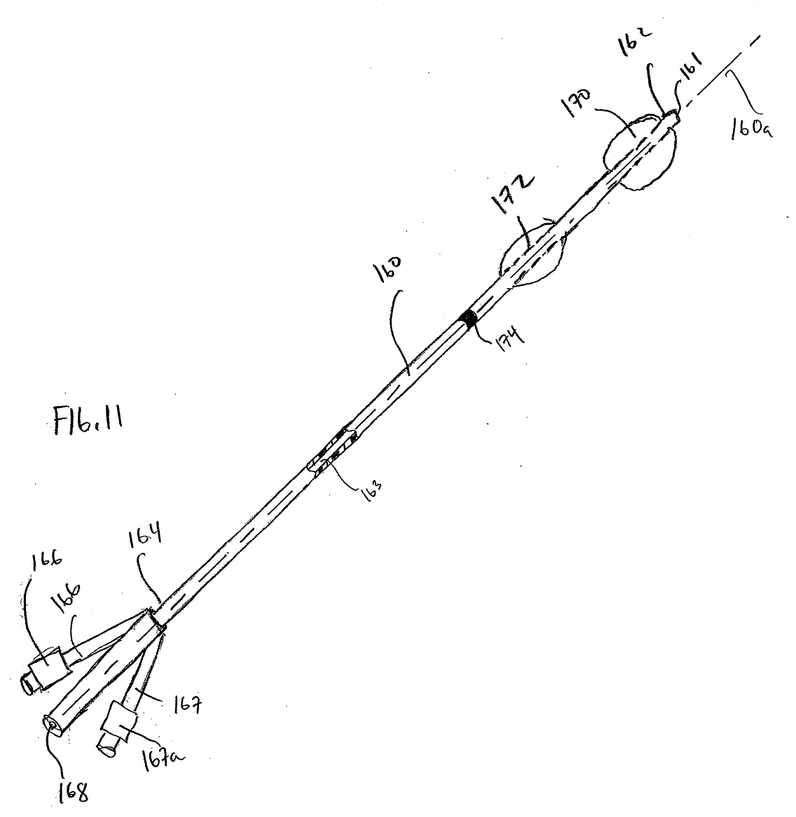

In operation, stent placement device 10 can be assembled and inserted into a patient using the following method steps shown in FIG. 10 and understood with reference to FIG. 1a. As shown in step 510 of FIG. 10, in embodiments where stent 20 includes a flared proximal end 24, the medical professional reduces flare 26 in proximal end 24 of stent 20. This step can be performed using an apparatus for reducing a flare in a stent that is known to those of skill in the art. For example, an apparatus for reducing flare 26 may include a mandrel shaft that is inserted through the stent 20 and a latch collar that retains the end of flare 26 of stent 20. The latch collar of the reducer holds the end of flare 26 stationary while the remainder of the stent 20 is rotated about the mandrel shaft to tighten the windings that form stent 20. Because flare 26 is at a greater diameter than the remainder of stent 20, the diameter of flare 26 is reduced when stent 20 is wound because the tip of flare 26 is prevented from moving and flare 26 has the lowest resistance to contraction. The winding of the remainder of stent 20 with respect to the flare 26 causes diameter of flare 26 to reduce until proximal end 24 of the stent 20 has the same outer and inner diameters as the remainder of the stent 20.

While stent 20 is maintained on the reducer, the stent is prevented from rotating with respect to the mandrel shaft and therefore the stent maintains its reduced orientation. With the reducer still connected to the stent 20, the physician inserts stent 20 into outer sheath 40. After stent 20 is inserted into shaft 40, the stent reducer is disconnected from stent 20. After the reducer is removed from stent 20, proximal end 24 remains in the reduced diameter while at room temperature. Additionally, outer sheath 40 substantially prevents proximal end 24 of stent 20 from expanding as the temperature of stent 20 is heated to match the patient's body temperature after stent 20 and outer sheath 40 are inserted into the patient.

When expanded as shown in FIG. 5, flared end 26 of the stent provides a surface that contacts the inner surface of the urethra or other conduit in the patient that stent 20 is inserted into. This contact aids in maintaining stent 20 in the required position after balloon catheter 60 and the remaining members of stent positioning device 10 are removed from the patient. As is discussed below, distal end 22 of stent 20 is preferably cut before use to modify the length of stent 20 to be the same as the distance between the bladder neck and the apex of the prostate.

Prior to the procedure, as is known to those of skill in the art and mentioned above, the medical professional may manipulate a urethral scope, or similar device, to determine the required length of stent 20 for accurate placement within the urethra or other flow path of the patient.

Specifically, the medical professional inserts the urethral scope into the patient until it has entered the bladder neck and then retracts the scope until it is positioned at the apex of the prostate. As mentioned above, stent 20 is preferably cut to be the same length as the distance between the bladder neck and the apex of the prostate before inserting stent 20 into the patient to allow for proper placement within the patient. Additionally, this procedure allows the medical professional to determine the amount of balloon catheter 60 that must to be inserted into the patient to allow balloon 70 to be inserted into the bladder, as discussed below.

After flare 26 has been reduced (with the stent reducer still connected to stent 20), stent 20 is inserted into outer sheath 40 as shown in step 520 of FIG. 10. As discussed above, flare 26 on proximal end 24 of stent 20 does not immediately expand to its flared diameter, but is restrained from expansion by the outer sheath 40. Outer sheath 40 is formed to be sufficiently flexible to allow outer sheath 40 to be removed from surrounding stent 20, including the flare 26, when performing step 570, discussed below.

After stent 20 is inserted into outer sheath 40, distal end 34 of stent pusher 30 is inserted into the aperture 48 in proximal end 44 of outer sheath 40. Stent pusher 30 can be urged into outer sheath 40 to move stent 20 toward distal end 42 of outer sheath 40. Stent 20 is correctly positioned within the outer sheath 40 when the positive stop 36 on the stent pusher 30 contacts the proximal end 44 of outer sheath 40. Alternatively, in embodiments that include a reference mark on stent pusher 30, stent 20 is correctly positioned when the reference mark is positioned at proximal end 44 of outer sheath 40. When stent 20 is in the selected position near distal end 42 of outer sheath 40, stent pusher 30 is removed from outer sheath 40 and discarded, as shown in Step 540.

In some embodiments, balloon catheter 60 is inserted into cavity 56 in stent positioner 50 when the stent placement device 10 is manufactured and packaged for sale. In other embodiments, balloon catheter 60 is inserted into cavity 56 of stent positioner 50 at this point in the procedure.

Balloon catheter 60 and stent positioner 50 are inserted into proximal end 44 of outer sheath 40, as shown in step 550. Balloon catheter 60 and stent positioner 50 are inserted into outer sheath 40 until balloon catheter 60 extends all the way through lumen 25 of stent 20 and through distal end 42 of outer sheath 40 (as shown in FIG. 2). Additionally, stent positioner 50 is selectively inserted into outer sheath 40 until distal end 52 of stent positioner 50 contacts proximal end 24 of stent 20.

Preferably, as shown in FIGS. 1a and 8a, clip 80 may be positioned to surround stent positioner 50 and balloon catheter 60 close to proximal ends 56, 64 of both members. Clip 80 preferably includes a first locked position (shown in FIG. 8a) and a second unlocked position (shown in FIG. 1a). When clip 80 is in the locked position, clip 80 applies a compressive force on stent positioner 50 and balloon catheter 60 to maintain balloon catheter 60 and stent positioner 50 in the selected position within outer sheath 40. While in the locked position, flow may be impeded through the balloon catheter 80 lumen 63 between the distal and proximal ends 62, 63, as well as through the lumen that connects the inflation/deflation port 66 with the balloon 70. While in the unlocked position, flow is not impeded through the various lumens within balloon catheter 60.

Clip 80 is maintained in the unlocked position when stent positioning device 10 is inserted into the patient in accordance with step 570 (discussed below). In embodiments including penile meatus dilator 90 and dilator sheath 96, meatus dilator 90 and dilator sheath 96 are inserted into patient in accordance with step 560. As discussed above, tapered distal end 92 of meatus dilator 90 is inserted into the patient's urethra within the penile meatus. The tapered distal end 92 provides for a gradual expansion of the urethra, which provides greater comfort for the patient than inserting the stent insertion device 10 directly into the patient's urethra. After distal end 92 fully inserted into the patient's urethra, dilator sheath 96 is inserted into the urethra. Dilator sheath 96 is approximately the same diameter as outer sheath 40, which allows stent insertion device 10 to be inserted into the patient's urethra in accordance with step 570, below, with minimal discomfort. After dilator sheath 96 is inserted into the patient's urethra, meatus dilator 90 is removed and discarded.

As shown in step 570 of FIG. 10, balloon catheter 60 and other assembled components of stent positioning device 10 are inserted into the urethra of the patient until distal end 62 and balloon 70 extend into the patient's bladder. In embodiments including penile meatus sheath 96, stent positioning device 10 is inserted through penile meatus sheath 96. In other embodiments, distal ends of outer sheath 40 and balloon catheter 60 are initially inserted directly into patient's urethra in their penile meatus. Because the medical professional determined before the procedure the distance between the apex of the prostate and the bladder, the medical professional selectively inserts the stent positioning device 10 until distal end 62 and balloon 70 of balloon catheter 60 enters the bladder by observing the length of balloon catheter 60 that has entered the patient. After stent positioning device 10 is inserted into the patient, dilator sheath 96 may be removed and discarded.

According to step 580 of FIG. 10, balloon 70 is inflated as best shown in FIG. 2. Initially in embodiments where clip 80 is provided, clip 80 is maintained in the unlocked position to allow liquid for flow through the lumens within the balloon catheter 60. As discussed above, a syringe (not shown) or other source of pressurized fluid is connected to inflation/deflation port 66 to inject a fluid into balloon catheter 60. When fluid is pumped into balloon catheter 60, it travels through a second lumen (not shown) until it reaches balloon 70, which serves as a reservoir for the fluid. The fluid is prevented from escaping balloon catheter 60 because inflation/deflation port 66 is capped shut or otherwise closed, as is understood by those of skill in the art. In other embodiments, inflation/deflation port can additionally be formed with a check valve that prevents flow out of balloon catheter 60 through the port (until the check valve is overridden by the medical professional). Therefore, as additional fluid is pumped into balloon catheter 60, the pressure within the balloon 70 increases causing expansion.

Eventually, balloon 70 expands to its rated volume, and the medical professional discontinues the addition of fluid to balloon catheter 60. It is important that the medical professional not over-inflate balloon 70 by adding excessive fluid above the specified volume of balloon catheter 60 to avoid bursting balloon 70 due to excessive pressure, or leakage from balloon 70. As is known to those of skill in the art, saline solution is preferably used as an inflation fluid, so leakage or failure of balloon 70 is not a significant health risk to the patient.

After balloon 70 is inflated, the medical professional slowly retracts the balloon catheter 60 (and associated 50) from the patient's urethra until the medical professional feels an obstruction or resistance to further withdrawal of balloon catheter 60. This obstruction is due the proximal end of the inflated balloon 70 being prevented from entering the urethra, because the diameter of the urethra is significantly smaller than inflated balloon 70. At this point stent 20 is positioned in the predetermined location within patient. Normally, stent 20 is positioned so that distal end 22 of stent 20 extends 2-5 mm into the bladder neck.

In embodiments including the radiopaque marker 43 on outer sheath 40 or one more radiopaque markers 74a, 74b on balloon catheter 70, the physician monitors the position of the radiopaque markers as discussed above. As discussed above, the indication of the position of the radiopaque markers within the patient provides an independent indication that that stent 20 is in its correct position.

Next, the medical professional transfers clip 80 to the locked position, which substantially prevents relative movement between balloon catheter 60 an stent positioner 50 to maintain stent 20 in the selected position within the patient. While maintaining the retraction on balloon catheter 60, the medical professional then removes outer sheath 40 from the placement device.

Outer sheath 40 is removed from stent positioning device 10 and the patient. Preferably, the medical professional removes outer sheath 40 from the patient by holding and pulling arms 45, 46 apart so that outer sheath 40 develops a tear along the longitudinal axis of outer sheath 40. While pulling arms 45, 46 apart, the medical professional simultaneously pulls arms 45, 46 of outer sheath 40 away from the patient, which provides a force to pull sheath out of the patient's orifice. As discussed above, outer sheath 40 is manufactured from a flexible material that allows outer sheath 40 to be pulled over the flare 26, which likely has expanded to the outer diameter of the urethra by the time step 590 is performed. Eventually, outer sheath 40 will be entirely removed from the patient and can be discarded. In embodiments with weakened region 49 formed on outer sheath 40, the outer sheath 40 can be easily removed by tearing outer sheath 40 along weakened region 49, or multiple weakened regions 49.

As shown in step 590 of FIG. 10, clip 80 is transferred to the unlocked position and stent positioner 50 and clip 80 are removed from the patient. At this point, only the balloon catheter 60 with inflated balloon 70 and stent 20 remain within the patient.

Depending on the specific medical procedure used in conjunction with stent 20, balloon catheter 60 is retained within the patient for varying times. In embodiments when the stent 20 is used during and after minimally invasive therapies for BPH, balloon catheter 60 is retained within the patient until the patient's urine is free of blood. Through past experience of patients being treated for BPH, typical times until patients have blood-free urine are between 12-24 hours. Obviously, the medical professional should determine when to remove balloon catheter 60 from the patient based on the patient's symptoms, medical history, and the medical professional's experience and training.

Finally, when the medical professional has determined that it is appropriate to remove balloon catheter 60, the medical professional removes balloon catheter 60 from the patient in accordance with step 600 of FIG. 10. First, the medical professional removes the cap or similar device from the inflation/deflation port 66, or otherwise allows fluid to escape from balloon 70 to the environment. In embodiments that include a check valve 67 on inflation/deflation port 66, the medical professional overrides the check valve 67. When sufficient fluid has drained, balloon 70 deflates due to the release of fluid pressure from within the balloon. After balloon 70 deflates, balloon catheter 60 can be slowly withdrawn from the patient. After sufficient removal of balloon catheter 60, distal end 62 is no longer inserted within stent 20. Stent 20 is retained in position because flare 26 engages the inner wall of the urethra (or other flow path in the patient), allowing stent 20 to maintain the urethra open to allow the unobstructed flow of urine. Finally, balloon catheter 60 is fully removed from the patient and discarded, leaving stent 20 in position within the urethra.

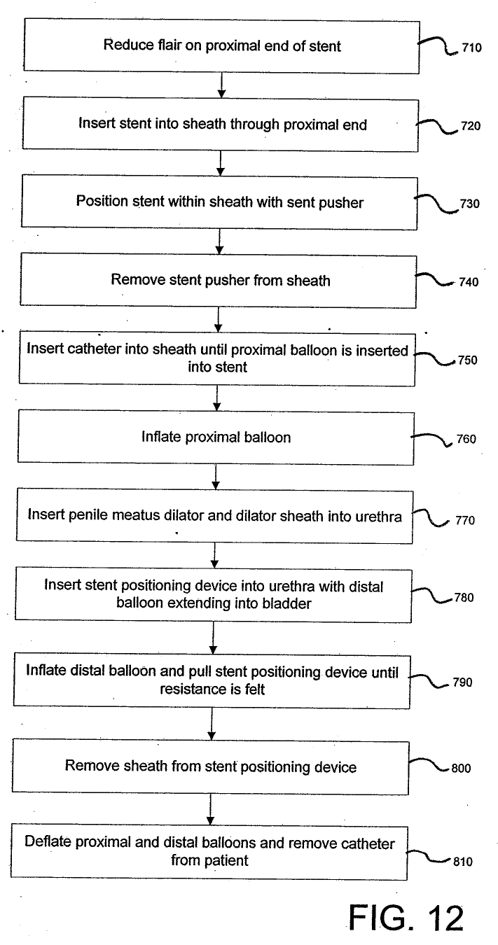

A second embodiment of stent positioning device 100 is provided and shown in FIGS. 11 and 12. This embodiment can be used in the same situations and medical circumstances as the embodiment described above. This embodiment includes many of the same components discussed above, including stent 20, stent pusher 30, and outer sheath 40. This embodiment additionally includes an alternate balloon catheter 160 that is shown in FIG. 11. Balloon catheter 160 includes a distal end 162 and a proximal end 164.

Balloon catheter 160 includes distal balloon 170 located at distal end 162 and proximal balloon 172 located proximally of distal balloon 170 along the longitudinal axis of balloon catheter 160. In some embodiments, a radiopaque marker 174 is provided rearwardly of proximal balloon 172 along the length of balloon catheter 160, or in another convenient location on balloon catheter 160. This radiopaque marker 174 is formed and detected in the same manner as discussed above in the first embodiment. Balloon catheter 160 additionally includes two inflation/deflation ports, a first inflation/deflation port 166 that is connected to distal balloon 170 through an inflation lumen (not shown) located within the balloon catheter 160 and a second inflation/deflation port 167 that is connected to proximal balloon 172 through a second inflation lumen (not shown) or vice-versa. Each of the first and the second inflation/deflation ports 166, 167 may include check valves 166a, 167a in some embodiments that prevent flow out of balloon catheter 160 from each of the balloons 170, 172. In embodiments that do not include check valves 166a, 167a, backflow of fluid from the balloons 170, 172 is prevented with a cap or a similar device. Each of first and second inflation/deflation ports 166, 167 are connected to proximal end 164 of balloon catheter 160. Inflation/deflation ports 166, 167 are preferably provided with Luer lock style valves to accept a source of pressurized working fluid from a syringe or other pressurized fluid source as is known in the art. Alternatively, the valves 166, 167 may be solid plugs that are opened for inflation or deflation using a syringe.

Balloon catheter 160 includes an inlet port 161 on distal end 162 that is connected with a drainage port 168 on proximal end 164 through a lumen 163 within balloon catheter 160 to allow for liquid flow through balloon catheter 160.

In operation, the stent positioning device 100 can be assembled and used with a patient using the following method steps (shown in flowchart form in FIG. 12). Steps 710, 720, 730, and 740 correspond to the respective previous steps 510, 520, 530, and 540 described above, and are not discussed in detail here.

After step 740 is completed, balloon catheter 160 is inserted into the opening in proximal end 44 of sheath 44, as shown in step 750 of FIG. 12. Balloon catheter 160 is advanced through the internal volume of outer sheath 40 toward distal end 42 until proximal balloon 172 is inserted through lumen 25 of stent 20. When proximal balloon 172 is inserted through lumen 25, proximal balloon 172 is inflated by injecting pressurized fluid into the inflation/deflation port 166 connected with proximal balloon 172, as shown in step 760. After the rated amount of fluid is injected into proximal balloon 172, it expands and contacts lumen 25 of stent 20, substantially eliminating any potential relative movement between stent 20 and balloon catheter 160. In other words, stent 20 is maintained stationary with respect to balloon catheter 160 when stent positioning device 100 is inserted into the patient until distal balloon 170 is inserted into the patient's bladder.

Similar to the embodiment discussed above, in embodiments including penile meatus dilator 90 and dilator sheath 96, meatus dilator 90 and dilator sheath 96 are inserted into patient in accordance with step 770. As discussed above, tapered distal end 92 of meatus dilator 90 is inserted into the patient's urethra within the penile meatus. The tapered distal end 92 provides for a gradual expansion of the urethra, which provides greater comfort for the patient than inserting the stent insertion device 10 directly into the patient's urethra. After distal end 92 fully inserted into the patient's urethra, dilator sheath 96 is inserted into the urethra. Dilator sheath 96 is approximately the same diameter as outer sheath 40, which allows stent insertion device 10 to be inserted into the patient's urethra in accordance with step 780, below, with minimal discomfort. After dilator sheath 96 is inserted into the patient's urethra, meatus dilator 90 is removed and discarded.

As shown in Step 780 of FIG. 12, distal end 162 of balloon catheter 160 and the remaining components of stent positioning device 100 are inserted into an orifice of the patient, which is the urethra within the penile meatus in the embodiments described in detail here. The distal balloon 170 and stent 20 are precisely positioned within the patient after the medical professional performs the steps discussed above to measure the required distance for insertion of balloon catheter 60 (in this embodiment balloon catheter 160) and to measure and cut stent 20 to the required length.

As shown in step 790 of FIG. 12, outer sheath 40 is removed from the patient after distal balloon 170 enters the bladder. The procedure for removal of outer sheath 40 is discussed above. In embodiments where outer sheath 40 is provided with at least one weakened region 49, outer sheath 40 can be torn along weakened region 49 to aid in the removal of outer sheath 40 from the patient. At the completion of step 790, only the balloon catheter 160 and stent 20 remain within the patient.

As discussed above with step 580 and shown in FIG. 12 as step 800, after distal balloon 170 enters the bladder, distal balloon 170 is inflated by injecting pressurized fluid into first inflation port 166 of balloon catheter 160. After distal balloon 170 is inflated the medical professional pulls balloon catheter 160 rearwardly until resistance is felt. At this position, distal balloon 170 is at the bladder neck, and stent 20 preferably extends into the bladder by 2 to 5 mm.

Depending on the medical procedure used in conjunction with stent positioning device 100 to insert stent 20 into the selected position within the patient, balloon catheter 160 is retained inserted into the patient for varying times. When the medical professional has determined that it is appropriate to remove balloon catheter 160, the medical professional removes balloon catheter 160 from the patient in accordance with step 810 of FIG. 12. First, the medical professional removes the cap or similar device connected to inflation/deflation ports 166, 167 on proximal end 164 of balloon catheter 160. In embodiments with check valves 166a, 167a located within inflation/deflation ports 166, 167, the medical professional overrides the check valves 166a, 166b. This allows fluid to flow from distal and proximal balloons 170, 172 to the environment, which deflates them due to the release of fluid pressure from within balloons 170, 172. After balloons 170, 172 are deflated, balloon catheter 160 is slowly withdrawn from the patient. After sufficient removal of balloon catheter 160, it is no longer is inserted within lumen 25 of stent 20. Stent 20 is retained in position because flare 26 engages the inner wall of the urethra (or other flow path in the patient), which maintains the stent 20 selectively positioned to maintain the urethra open to allow the unobstructed flow of urine. Finally, balloon catheter 160 is fully removed from the patient and discarded, leaving stent 20 in position within the urethra.

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein.

What is claimed is:

1. A method for inserting a stent in a patient, comprising the steps of:
(a) inserting a stent into an outer sheath;
(b) inserting a catheter and a stent positioner into the outer sheath, until the catheter extends through a lumen in the stent and the distal end of the stent positioner contacts a proximal end of the stent;
(c) positioning the stent positioner within the outer sheath so a distal end of the stent is proximal of a balloon on the catheter along a longitudinal axis of the catheter;
(d) inserting the outer sheath, the catheter, the stent, and the stent positioner into a patient and selectively withdrawing the outer sheath;
(e) inflating the balloon in the catheter;
(f) positioning the stent within the patient; and
(g) selectively withdrawing the positioning shaft from the patient.

2. The method of claim 1 further comprising the step of inserting a meatus dilator and a meatus dilator sheath into the patient prior to inserting the outer sheath, the catheter, the stent, and the stent positioner into the patient.

3. The method of claim 2 further comprising the step of removing the meatus dilator prior to inserting the outer sheath, the catheter, the stent, and the stent positioner into the patient.

4. The method of claim 1 further comprising the step of withdrawing the catheter from the patient.

5. The method of claim 4 wherein the catheter is withdrawn from the patient when a predetermined condition occurs.

6. The method of claim 1 wherein the outer sheath includes a weakened region.

7. The method of claim 1 wherein the stent positioner includes a cavity.

8. The method of claim 7 wherein a cross-section of the stent positioner is substantially shaped as a C.

9. The method of claim 8 wherein a proximal end of the stent is formed with a greater diameter than a diameter of the distal end of the stent, and further comprising the step of reducing the diameter of the proximal end to a diameter that is substantially the same as a diameter of the distal end the stent prior to the step of inserting the stent into the sheath.

10. The method of claim 1 further comprising the step of retaining the catheter within the positioning shaft with a clip.

11. A stent positioning device comprising:
(a) a catheter including a distal end and a proximal end and with a balloon positioned on the distal end,
(b) a stent with a distal end and a proximal end, wherein the stent may surround the catheter and be positioned rearwardly of the balloon along a longitudinal axis of the catheter when the stent positioning device;
(c) a stent positioner formed to substantially surround the catheter and be positioned proximally of a proximal end of the stent when the stent positioning device is assembled; and
(d) an outer sheath surrounding the stent, the stent positioner, and the majority of the catheter when the stent positioning device is assembled.

12. The stent positioning device of claim 11 wherein the stent positioner includes a cavity.

13. The stent positioning device of claim 12 wherein a cross-section of the stent positioner is substantially shaped as a C.

14. The stent positioning device of claim 13 wherein the proximal end of the stent is formed with a greater diameter than the distal end of the stent.

15. The stent positioning device of claim 11 further comprising a meatus dilator and a dilator sheath surrounding the meatus dilator, wherein the outer sheath is insertable through the dilator sheath when the meatus dilator is removed from the dilator sheath.

16. The stent positioning device of claim 11 wherein the outer sheath includes a radiopaque marker.

17. The stent positioning device of claim 11 wherein the balloon catheter includes a radiopaque marker.

18. A stent positioning device comprising:
(a) a catheter including a distal end and a proximal end, with a first balloon positioned on the distal end and a second balloon positioned proximally of the first balloon along a longitudinal axis of the catheter;
(b) a stent with a distal end and a proximal end, the stent surrounding the second balloon of the catheter when the stent positioning device is assembled; and
(c) an outer sheath surrounding the stent and the majority of the catheter when the stent positioning device is assembled.

19. The stent positioning device of claim 18 wherein the catheter further comprises a first inflation port extending from the proximal end of the catheter and in fluid communication with the first balloon and a second inflation port extending from the proximal end of the catheter and in fluid communication with the proximal balloon.

20. The stent positioning device of claim 19 wherein the first and the second balloons may be independently inflated.

21. The stent positioning device of claim 20 wherein the stent is retained in a selected position with respect to the catheter when the second balloon is inflated.

22. The stent positioning device of claim 18 further comprising a meatus dilator and a dilator sheath surrounding the meatus dilator, wherein the outer sheath is insertable through the dilator sheath when the meatus dilator is removed from the dilator sheath.

23. The stent positioning device of claim 18 wherein the catheter includes a radiopaque marker.
